(19) United States
(12) Patent Application Publication
(10) Pub. No.: US 2010/0267679 A1
(43) Pub. Date: Oct. 21, 2010

(54) Pharmaceutical combination of medication to be used by patients for the purpose of preventing or reducing the evolution of atherosclerosis and thus preventing coronary heart disease and cerebro-vascular accidents.

(76) Inventor: JACQUES BENOIT, (US)

Correspondence Address: JACQUES BENOIT, 7013 JEAN MILOT, MONTREAL, QC, H1M3G4, CA

(21) Appl. No.: 12/386,165

(22) Filed: Apr. 15, 2009

(51) Int. Cl. A61K 31/60; A61K 31/41; A61K 31/40; A61K 31/366; A61K 31/137

(52) U.S. Cl. 514/165; 514/381; 514/423; 514/460; 514/649

(57) ABSTRACT

Heart diseases such as myocardial infarction and cerebro-vascular diseases remain major causes of death. The underlying disease process involved is usually atherosclerosis. The cause of human atherosclerosis is unknown. At best we know of risk factors. The purpose of the present invention is to act on the risk factors so as to reduce or prevent the development or evolution of atherosclerosis and reduce coronary heart diseases and cerebro-vascular diseases. That treatment is easy to use, effective, safe, and readily available. The present invention describes a pharmaceutical combination of medication for that very purpose. The combination comprises the following ingredients: (1) an angiotensin converting enzyme blocker (ACE), (2) a Beta adrenergic blocker, (3) an antiplatelet agent, (4) a Statin, (5) a calcium channel blocker.

Some definitions need to be clarified.

Arteriosclerosis is a general term describing any hardening (and loss of elasticity) of medium or large arteries. Arteriolosclerosis is any hardening (and loss of elasticity) of arterioles (small arteries). Atherosclerosis is a hardening of an artery specifically due to an atheromatous plaque. Therefore, atherosclerosis is a form of arteriosclerosis: it is the late stage of arterioclerosis

### FIELD OF THE INVENTION

Hundreds of thousands of people die from myocardial infarction annually. The cost of medication and/or surgical interventions on survivors of a myocardial infarction is huge and the loss in human productivity is also very high. That is why a medication that can effectively prevent the evolution of atherosclerosis and thus reduce the incidence of coronary heart disease and myocardial infarction is needed. While atherosclerosis in the cerebral arteries can often lead to thrombosis, haemorrhages, embolism with destruction of parts of the cerebrum resulting in paralysis, paresis, loss of orientation, dysphasia, apraxia and total dependence on intensive nursing the whole resulting in billions of dollars in loss of productivity and societal costs for support of the invalid person. A medication that could reduce the evolution of atherosclerosis in the cerebral arteries would also be very helpful. It is obvious that the medications that can reduce the evolution of atherosclerosis will affect both the coronary and cerebral arteries and reduce the incidence of myocardial ischemia/infarction and cerebral ischemia/infarction.

### BACKGROUND TO THE INVENTION

The purpose of the present invention is to incorporate various pharmalogical agents that will act on the risk factors of atherosclerosis so as to reduce or prevent its development or evolution and hence reduce the incidence of the coronary heart disease and cerebro vascular diseases. That treatment is easy to use, effective, safe, and readily available. The present invention describes a pharmacological combination of medication to reduce the risk of coronary heart disease and cerebro-vascular diseases by acting on multiple levels of known causative processes or associated risk factors, those being:

(1)-Hypertension, (2)-Hyperlipidemia, mainly high levels of low density lipoproteins (LDL), high levels of tri-glycerides (TG), and low levels of high density lipo proteins (HDL), (3)-coronary vaso constriction, (4)-myocardial instability+/−arrhythmias, (5)-platelet aggregation with clot formation and coronary thrombosis or cerebro-vascular thrombosis or embolism.

### SUMMARY OF THE INVENTION

Atherosclerosis is the leading cause of heart diseases and cerebro-vascular diseases, The cause(s) of the disease process are unknown but the risk factors are known. The present invention introduces a medication that includes a group of five different pharmacological agents that have a positive action on the risk factors. That treatment is easy to use, effective, safe, and readily available. The present invention is a medication that comprises the following ingredients: an angiotensin converting an enzyme blocker (ACE) such as Linisopril 10 mgs, a Beta adrenergic blocker such as Metoprolol 50 mgs, an antiplatelet agent such as acetyl salycilic acid 80 mgs, a Statin such as Lovastatin 40 mgs, a Calcium channel blocker such as amiodipine besylate 2.5-5 mgs. The dosage of the medications in the combination can vary.

### DETAILED DESCRIPTION OF THE INVENTION

Atherosclerosis is an insidious disease process syndrome affecting arterial blood vessels. It is a chronic inflammatory response in the walls of arteries, in large part due to the accumulation of macrophage white blood cells and promoted by low density (especially small particle) lipoproteins (plasma proteins that carry cholesterol and triglycerides) without adequate removal of fats and cholesterol from the macrophages by functional high density lipoproteins (HDL). As a result a chronic inflammatory process is initiated and maintained in the wall of affected arteries. It is commonly referred to as a hardening or furring of the arteries. It is caused by the formation of multiple plaques in the arteries. As the inflammation process evolves, there is a progressive accumulation of cholesterol crystals, dead macrophages and other white blood cells, progressively there is a process of calcium deposit in the sub intimal and medial space of the arterial wall. That process of calcification is a sign of advanced atherosclerosis. As the process evolves, the lumen of the artery is reduced in an important fashion and the arterial flow is reduced according to Poiseuille's law.

FLOW RATE=((P1−P2)*R̂4*PI)/(8*L*n)

P1=initial pressure
P2=final pressure

### PI=3.141516

R=radius of pipe (artery)
L=length of pipe (artery)
n=constant for specific fluid.

If the radius (R) and all factors remain constant, the percent reduction in flow can be calculated the following way:

Remaining Flow Rate as a percentage=(1−% reduction in radius)̂4*100

For instance a reduction of 20% of the arterial lumen (by an atherosclerotic plaque) will reduce the flow to 41% of the original flow. A reduction of the arterial lumen by 50% will reduce the flow to 6.5%. A reduction of the arterial lumen by 70% will reduce the flow to 0.8%

As can be seen even a slight reduction in the radius of the lumen will have a massive impact on reducing the blood flow.

As a consequence of the reduced arterial perfusion of the myocardium (heart muscle) Myocardial ischemia is a condition in which there is oxygen deprivation to the heart muscle accompanied by inadequate removal of metabolites because of reduced blood flow or perfusion. Myocardial ischemia can occur as a result of increased myocardial oxygen demand, reduced myocardial oxygen supply, or both. In the presence of coronary obstruction, an increase of myocardial oxygen requirements caused by exercise, tachycardia, or emotion leads to a transitory imbalance. This condition is frequently termed demand ischemia and is responsible for most episodes of chronic stable angina. In other situations, the imbalance is caused by acute reduction of oxygen supply secondary to increased coronary vascular tone (ie, coronary vasospasm) or by marked reduction or cessation of coronary flow as a result of platelet aggregates or thrombi. This condition, termed supply ischemia, is responsible for myocardial infarction (MI) and most episodes of unstable angina (UA). In many circumstances, ischemia results from both an increase in oxygen demand and a reduction in supply.

Myocardial ischemia most often develops as a result of reduced blood supply, due to atherosclerotic plaques located in the coronary arteries. The plaques initially allow sufficient blood flow to match myocardial demand. These areas of narrowing may become clinically significant and precipitate angina when myocardial demand increases. Angina that is reproduced by exercise, eating, and/or stress and is subsequently relieved with rest and without recent change in frequency or severity of activity necessary to produce symptoms is called chronic stable angina; patients usually learn to recognize their symptoms. Over time, the plaques may thicken and rupture, exposing a thrombogenic surface upon which platelets aggregate and thrombi form. The lumen of the coronary artery is further reduced and more and more platelets aggregate with the resulting clot formation. The consequence is a marked reduction in coronary blood flow, this syndrome is called “acute coronary syndrome”, resulting in a myocardial syndrome. The symptoms felt by the patient at this time of an impending myocardial infarct (MI) is a retro-sternal chest pain, constrictive in nature, Variants exist regarding the location and type of pain: the pain is often accompanied by a sense of fatigue, weakness, and a cold sweat. A myocardial infarct (MI) involves destruction of sections of the heart muscle with all its complications, i.e., pump failure, arrhythmias, heart wall rupture, instant death.

A similar process of atherosclerosis may take place in the cerebro vascular arteries. With very similar damages to the arteries and leading clinically to cerebro vascular accidents such as cerebral thrombosis, cerebral haemorrhages, cerebral embolism resulting in necrosis in the cerebrum and paralysis, paresis, loss of orientation, dysphasia, apraxia and total dependence on intensive nursing the whole resulting in billions of dollars in loss of productivity and societal costs for support of the invalid person. Often The first clinical presentation of cerebral atherosclerosis is often a Transient Ischemic Attack (TIA). Physiologically as a thrombus forms and blocks either a coronary or cerebral artery there is a massive reduction of blood flow and a reactive arterial wall spasm which further reduces blood flow.

Risk factors for the development of atherosclerosis are many. Clinically it is important to address the reversible risk factors, such as diabetes mellitus, hypertension, cigarette smoking, dyslipidemia (elevated LDL, or elevated TG or reduced HDL), obesity (central type), sedentary life style, cardiac arythmias. A large number of other minor risk factors exist that may be addressed clinically on an individual basis.

Some well known non-pharmacological methods to prevent atherosclerosis are well known. For instance there is a medical consensus that diet modifications, such as reducing body weight so as to obtain a Body Mass Index (BMI) between 20 and 25, reducing fat intake and increasing vegetable and fruit intake can also be of help. Reducing sedentarity by increasing physical activities such as sports and reducing stress have also been shown to be of help.

### Details on the Agents Contained in the Invention

1—The pharmaceutical combination according to claim 1 comprises an angiotensin converting enzyme blocker (ACE) such as Linisopril.

Angiotensin converting enzyme blockers (ACE) are commonly used for the prevention of cardiovascular disorders, for the treatment and prevention of hypertension, for the prevention and treatment of congestive heart failure CHF, for the prevention and treatment of left ventricular dysfunction, for the prevention of nephropathy in diabetes mellitus.

ACE inhibitors lower arteriolar resistance and increase venous capacity, increase cardiac output and cardiac index, stroke work and volume, lower renovascular resistance, and lead to increased natriusesis (excretion of sodium in the urine).

ACE inhibitors were initially used to treat hypertension. However they have been shown to be effective for the prevention of diabetic nephropathy, for the prevention and treatment of congestive heart failure, and for the prophylaxis of cardiovascular events such as myocardial infarction. Such use is justified because ACE inhibitors improve clinical outcomes, independent of their blood pressure lowering effect.

Angiotensin Receptor Blocking Agent (ARB) selectively and competitively block the binding of angiotensin II to the AT1 subtype receptor in vascular smooth muscle and the adrenal gland, preventing AT II-mediated vasoconstriction, aldosterone synthesis and secretion, and renal reabsorption of sodium. The end result is vasodilation, increased excretion of sodium and water, a reduction in plasma volume, and a reduction in blood pressure.

2—The pharmaceutical combination according to claim 1 comprises a beta adrenergic blocking agent such as metoprolol. Basically Beta adrenergic blockers are used for various indications, such as the prevention of angina (myocardial hypoxia), prevention and treatment of cardiac arrhythmias and hypertension. They can also be used successfully in cardioprotection.

Beta adrenergic blockers have an antihypertensive effect. The antihypertensive mechanism appears to involve reduction in cardiac output (due to negative chronotropic and inotropic effects), there is also a reduction in renin release from the kidneys, and if the beta adrenergic blocker crosses the blood brain barrier (such as Propanolol) there is a central nervous system effect to reduce sympathetic activity.

Antianginal effects result from negative chronotropic and inotropic effects, which decrease cardiac workload and oxygen demand. Negative chronotropic properties of beta adrenergic blockers permit the heart rate control.

The antiarrhythmic effects of beta adrenergic blockers arise from sympathetic nervous system blockade, resulting in depression of sinus node function and atrioventricular node conduction, and prolonged atrial refractory periods. Some can also prolong action potential duration through potassium channel blockade (Sotalol is such an agent).

Blockade of the sympathetic nervous system reduces the release of renin wich leads to reduced aldosterone via the renin angiotensin aldosterone system with a resultant decrease in blood pressure due to decreased sodium and water retention.

3—The pharmaceutical combination according to claim 1 comprises an antiplatelet agent. Antiplatelet drugs decrease platelet aggregation and inhibit thrombus formation. They are effective in the arterial circulation. They are widely used in primary and secondary prevention of thrombotic cerebro-vascular or cardiovascular disease.

4. The pharmaceutical combination according to claim 1 comprises a Statin.

### Pharmacology:

Statins act by inhibiting the enzyme HMG-CoA reductase, the enzyme controlling the first committed step of sterol (cholesterol) synthesis, in the liver. Because statins are similar to HMG-CoA on a molecular level they take the place of HMG-CoA in the enzyme and reduce the rate by which it is able to produce mevalonate, the next molecule in the metabolic path that eventually produces cholesterol, as well as a number of other compounds. Inside the liver cell, other enzymes of the protease class sense the decreased level of cholesterol produced. In response, they cleave a protein called “membrane-bound sterol regulatory element binding protein”, which then responds by migrating to the nucleus to increase production of various other proteins and enzymes, including the LDL receptor. The LDL receptor then relocates to the cell membrane of the liver cell, and binds to passing low density lipoprotein and very low density lipoprotein particles (both containing cholesterol in the undesired form). LDL and VLDL enter the liver and are digested.

### Indications and Uses:

Statins, the most potent cholesterol-lowering agents available. On average they lower LDL cholesterol (so-called “bad cholesterol”) by 1.8 mmol/l. This translates into a 60% decrease in the number of cardiac events (heart attack, sudden cardiac death) and a 17% reduced risk of stroke. It was generally recommended that patients try a lipid lowering diet and life style changes before using statins. Over time clinical indications for the use of statins have broadened.

Initial studies, such as the Scandinavian Simvastatin Survival Study (4S), supported the use of statins in secondary prevention for cardiovascular disease, or as primary prevention only when the risk for cardiovascular disease was significantly raised (as indicated by the Framingham risk score). Indications were broadened considerably by studies such as the Heart Protection Study (HPS), which showed preventative effects of statin use in specific risk groups, such as diabetics. The ASTEROID trial, published in 2006, using only a statin at high dose, achieved lower than usual target calculated LDL values and showed disease regression within the coronary arteries using intravascular ultrasonography.

Based on clinical trials, the National Cholesterol Education Program guidelines, and the increasing focus on aggressively lowering LDL-cholesterol, the statins continue to play an important role in both the primary and secondary prevention of coronary heart disease, myocardial infarction, stroke and peripheral artery disease.

In short, Statins have been shown to reduce blood levels of low density lipoproteins (LDL) and reduce the amount of cholesterol in the arterial wall. Statins have been shown to reduce atherosclerotic plaques and reduce the incidence of myocardial infarction in a population of patients suffering from established atherosclerosis (angina or post myocardial infarction) and in normal populations.

5—The pharmaceutical combination according to claim 1 comprising a Calcium channel blocker.

Calcium channel blockers are a class of drugs and natural substances which disrupt the conduction of calcium channels. It has effects on many excitable cells of the body, such as cardiac muscle, i.e. heart, smooth muscles of blood vessels and neurons. The main clinical usage of calcium channel blockers is to decrease blood pressure. It is for this action that they are used in individuals with hypertension.

Most calcium channel blockers decrease the force of contraction of the myocardium. This is known as the negative inotropic effect of calcium channel blockers. Many calcium channel blockers also slow down the conduction of electrical activity within the heart, by blocking the calcium channel during the plateau phase of the action potential of the heart. This results in a negative chronotropic effect resulting in a lowering of the heart rate. The negative chronotropic effects of calcium channel blockers make them a commonly used class of agents in individuals with atrial fibrillation or flutter in whom control of the heart rate is an issue.

### Mechanisms of Action:

Calcium channel blockers work by blocking voltage-gated calcium channels (VGCCs) in muscle cells of the heart and blood vessels. This prevents calcium levels from increasing as much in the cells when stimulated, leading to less muscle contraction. In the heart, a decrease in calcium available for each beat results in a decrease in cardiac contractility. In blood vessels, a decrease in calcium results in less contraction of the vascular smooth muscle and therefore an increase in blood vessel diameter, a phenomenon called vasodilation. Vasodilation decreases total peripheral resistance, while a decrease in cardiac contractility decreases cardiac output. Since blood pressure is in part determined by cardiac output and peripheral resistance, blood pressure drops. With a relatively low blood pressure, the afterload on the heart decreases; this decreases the amount of oxygen required by the heart. This can help improve symptoms of ischemic heart disease such as angina pectoris.

Phenylalkylamine calcium channel blockers such as Verapamil and Gallopamil are relatively selective for myocardium activity, they reduce myocardial oxygen demand and reverse coronary vasospasm, and are often used to treat angina. They have minimal vasodilatory effects compared with dihydropyridines. Their action is intracellular.

Benzothiazepine calcium channel blockers such as Diltiazem are an intermediate class between phenylalkylamine and dihydropyridines in their selectivity for vascular calcium channels. By having both cardiac depressant and vasodilator actions, benzothiazepines are able to reduce arterial pressure without producing the same degree of reflex cardiac stimulation caused by dihydropyridines.

### EXAMPLE

### Example 1

The invention medication may contain the following pharmaceutical agents: Linosopril 10 mgs, metoprolol 50 mgs, acetyl salycilic acid 80 mgs, lovastatin 40 mgs, amiodipine besylate 2.5 mgs. These agents are integrated in one tablet or capsule or syrup or other liquid dispensing agent. Normally the medication is taken once a day. The patient should be monitored by a physician on a regular basis.

### Example 2

The invention medication may contain the following pharmaceutical agents: Losartan 25 mgs, metoprolol 50 mgs, acetyl clopidogrel 50 mgs, lovastatin 40 mgs, amiodipine besylate 2.5 mgs. These agents are integrated in one tablet or capsule or syrup or other liquid dispensing agent. Normally the medication is taken once a day. The patient should be monitored by a physician on a regular basis.

### Example 3

The invention medication may contain the following pharmaceutical agents: Linosopril 8 mgs, Losartan 50 mgs, metoprolol 50 mgs, acetyl salycilic acid 80 mgs, lovastatin 40 mgs, amiodipine besylate 2.5 mgs. These agents are integrated in one tablet or capsule or syrup or other liquid dispensing agent. Normally the medication is taken once a day. The patient should be monitored by a physician on a regular basis.

### USE OF THE INVENTION

A physician may evaluate the risk of a patient having a myocardial infarction within 10 years using various scoring methods such as the Framingham risk evaluation score, the calcium coronary score (CCS) or any other suitable rating system. Using coronary calcium scores (CCS) to estimate the individual's “coronary artery calcium (CAC) age equivalent”. The accuracy of risk assessment could be improved by substituting CCS for age as a risk factor in Framingham risk scoring. Other risk factors have also been proposed recently such as the C Reactive Protein (CRP). At any rate it is a physician who will decide which patient will benefit best from treatment with the present invention.

The medication should be taken once a day, however it may also be taken more often if the invention is used at a lower dosage than the dosage described in the previous example.

If the patient presents specific risk factors that are not corrected by the invention specific medications may be added or the dosage of medications included in the invention may be increased. For instance a patient may be taking the invention medication once a day, yet his/her blood pressure remains uncontrolled then the treating physician may add a separate beta adrenergic blocker at 50 mg twice a day or even 100 mg twice a day if required. At the same time a calcium blocker may be added separately and if required a separate ACE blocker. It is a matter of medical practice. The point is to stress the fact that the invention is a basic medication that does not preclude the use of other medications.

When should the medication be started? The earlier the patient starts taking the medication, the earlier the protective action starts and the less are the damages caused to arteries by atherosclerosis and the longer the medication is taken the more important the regression of the atherosclerotic plaques, hence a reduction in the incidence of myocardial infarction or cerebro-vascular accidents.

The incidence of side effects is low and the potential side effects are also minimal when compared with the potential evolution of the process (a myocardial infarction+/−cardiac arrhythmias) in the absence of prompt treatment. Medically speaking the ratio of gain/risk benefit is highly positive.

While the preferred embodiments of the invention have been shown and described, modifications thereof can be made by one skilled in the art without departing from the spirit and teachings of the invention. The embodiments described herein are exemplary only, and are not intended to be limiting. Many variations and modifications of the invention disclosed herein are possible and are within the scope of the invention. Accordingly, the scope of protection is not limited by the description set out above, but is only limited by the claims herein made.

The embodiment of the invention in which an exclusive property or privilege is claimed are defined as follows:

1- A combination of medication used as either capsule, tablet, syrup, elixir or any other acceptable format.

2- The pharmaceutical combination according to claim 1 comprising an angiotensin converting enzyme blocker (ACE) such as Linisopril, or other angiotensin converting enzyme blocker selected from the group consisting of: Captopril, Zofenopril, Enalapril, Ramipril, Quinapril, Perindopril, Lisinopril, Benazepril, Fosinopril.
Alternatively the pharmaceutical combination according to claim 1 may comprise an Angiotensin Receptor Blocking Agent (ARB) such as losartan or other Angiotensin Receptor Blocking Agent (ARB) selected from the group consisting of: Valsartan, Telmisartan, Eprosartan, Irbesartan, Candesartan, Olmesartan. Alternatively the pharmaceutical combination according to claim 1 may contain both an Angiotensin Converting Enzyme Blocker (ACE) and an Angiotensin Receptor Blocking Agent (ARB).

3- The pharmaceutical combination according to claim 1 comprising the following: a beta adrenergic blocking agent such as metoprolol, or other beta adrenergic blocker selected from the group consisting of: acebutolol, alprenolol, amosulalol, arotinolol, atenolol, befunolol, betaxolol, bevantolol, bisoprolol, bopindolol, bucumolol, bufetolol, bufuralol, bunitrolol, buprandolol, butofilolol, carazolol, carteolol, carvedilol, celiprolol, cetamolol, cloranolol, dilevalol, epanolol, indenolol, labetalol, levobunolol, mepindolol, metipranolol, metoprolol, moprolol, nadolol, io nadoxolol, nebivalol, nipradilol, oxprenolol, perbutolol, pindolol, practolol, pronethalol, propranolol, sotalol, sufinalol, talindol, tertatolol, tilisolol, timolol, toliprolol, and xibenolol, and pharmaceutically acceptable salts or solvates thereof, or solvates of such salts.

4- The pharmaceutical combination according to claim 1 comprising an antiplatelet agent such as acetyl salycilic acid 80 mgs. Alternately the antiplatelet agent can be clopidogrel, or again the antiplatelet agent can be a combination of acetyl-salicylic acid (aspirin) and clopidogrel. The antiplatelet agent may also be selected from the following, ticlodipine, cilostazol, dipyridamole.

5- The pharmaceutical combination according to claim 1 comprising a Statin such as Lovastatin 40 mgs or other Statins selected from the group consisting of: Atorvastatin, Cerivastatin, Fluvastatin, Mevastatin, Pitavastatin, Pravastatin, Rosuvastatin, Simvastin.

6- The pharmaceutical combination according to claim 1 comprising a Calcium channel blocker such as amiodipine besylate 2.5 mgs, or other calcium channel blocker selected from the group consisting of: Amlodipine, Aranidipine, Azelnidipine, Barnidipine, Benidipine, Cilnidipine, Clevidipine, Efonidipine, Felodipine, Lacidipine, Lercanidipine, Manidipine, Nicardipine, Nifedipine, Nilvadipine, Nimodipine, Nisoldipine, Nitrendipine, Pranidipine.
